(19) United States
(12) Patent Application Publication
(10) Pub. No.: US 2024/0174957 A1
(43) Pub. Date: May 30, 2024

(54) APPARATUS AND PROCESS FOR CULTURING TISSUE

(71) Applicant: Meatable B.V., Leiden (NL)

(72) Inventors: Sina Dezfuli, Vlaardingen (NL); Duong Nguyen, Goteborg (SE); Sandra Ines Speicher, Delft (NL); Elyes Yaich, Rotterdam (NL); Denise Peeters, Rijswijk (NL)

(73) Assignee: Meatable B.V., Leiden (NL)

(21) Appl. No.: 18/551,202

(22) PCT Filed: Mar. 25, 2022
(86) PCT No.: PCT/NL2022/050165

§ 371 Date: Sep. 19, 2023

(30) Foreign Application Priority Data
Mar. 25, 2021 (NL) 2027836

(51) Int. Cl. C12M 3/00; C12M 1/12; C12M 1/34; C12M 1/42

(52) U.S. Cl. CPC C12M 21/08; C12M 25/14; C12M 35/02; C12M 35/04; C12M 41/12

(57) ABSTRACT

An apparatus for culturing muscle tissue comprises a chamber extending from a first end to a second end; a first anchor located within the chamber at the first end; a second anchor located within the chamber at the second end; an inlet; and an outlet. The chamber can have an expanding or cylindrical cross-section from each of the first end and the second end to a point between the first end and the second end. The first and second anchors are three-dimensional.

### FIELD OF THE INVENTION

The present invention relates to producing tissues from cells, and more specifically an apparatus and methods for production of muscle tissue.

### BACKGROUND

The term cultured meat, also called in vitro meat, lab-grown meat, cell-based meat, cultivated meat and synthetic meat is used to refer to muscle tissue grown from a cell or tissue culture as opposed to slaughtering animals to obtain the muscle tissue. This is desired to reduce the environmental impact of the agricultural system, as well as to address animal welfare issues and keep up with the growing demand for meat in the world.

The first attempts at cultured meat production have thus far used many of the same tissue engineering techniques used in regenerative medicine, producing small amounts of tissue in small bioreactor chambers with labor intensive processes.

### SUMMARY

According to a first aspect of the invention, an apparatus for culturing muscle tissue comprises a chamber extending from a first end to a second end,; a first three-dimensional anchor located within the chamber at the first end; a second three-dimensional anchor located within the chamber at the second end; an inlet; and an outlet. Such an apparatus can culture large pieces of muscle tissue between the three-dimensional anchors as the three-dimensional anchors allow for a plurality of attachment points for secure connections, allowing for more distance between then anchors than past systems and therefore larger muscle tissues.

In some embodiments, the chamber can have an expanding cross-section from each of the first end and the second end to a point between the first end and the second end. Such an apparatus with a chamber which has an expanding cross-section from each end to a middle point (for example, barrel or oval shaped) results in the ability to produce muscle tissue in large, desirable shapes when working with tissue which compacts. Having a chamber with an expanding cross-section to a middle point results in the muscle tissue grown in the chamber having a consistent, or even a convex shape between anchors after compaction, producing more muscle tissue and more closely mimicking muscle tissue produced in animals.

According to an embodiment, each of the first anchor and the second anchor have a biomimetic shape, for example, modelled after a tendon. Optionally, the three-dimensional anchor comprises an outer perimeter and a plurality of attachment paths extending from the outer perimeter. Further optionally, the outer perimeter is cylindrical, and/or the plurality of attachment paths do not align in the vertical direction. In further embodiments, the attachment paths may align in the vertical direction.

According to an embodiment, each three-dimensional anchor is an auxetic design. Auxetic design, as used herein, means that the anchor is formed of three dimensional building blocks which, when a force is applied in one direction results in an expansion in a perpendicular direction to the direction of the applied force. Optionally, the three-dimensional anchor is formed of a plurality of auxetic building blocks connected together. The building blocks could be, for example, butterfly shaped hexagon prisms connected at a center point which can deform into square or rectangular shapes. Further optionally, the anchor is a square or rectangular configuration with a plurality of building blocks connected together in at least two horizontal planes, each of the horizontal planes being connected to each other vertically. The use of relatively small auxetic building blocks providing small pores has been shown to result in a positive influence in the attachment of the tissue to the anchor, and the auxetic design allows for the use of these small building blocks in a repeated manner to build up the anchor to any size required which maintaining many attachment points and strength. The auxetic design provides for additional strength, even when subject to relatively high mechanical stimulation loads, with forces in one direction resulting in elongation in another.

In some embodiments, one or more of the anchors can be formed of a conductive material, for example metals (including alloys) such as titanium, ferritic steel, iron and carbon. Such a three-dimensional anchor design with a plurality of attachment paths create a plurality of pores in the anchors where the muscle tissue is able to wrap around and attach to many different points and sides of the attachment paths or auxetic building blocks and the perimeter of the anchors. This results in stronger connections between then anchors and muscle tissue, allowing for the growth of larger muscle tissue and for more mechanical stimulation, resulting in more efficient growth and muscle tissue quality in terms of texture, appearance and taste. The use of conductive material to form anchors allows for the direct connection and/or integration of one or more electrodes into the apparatus to provide electrical stimulation to chamber, to promote cell differentiation and tissue compaction as well as muscle tissue growth.

According to an embodiment, the chamber cross-section is circular, oval, rectangular or triangular. Other chambers could contemplate other designs as well, such as octagon or another polygonal cross-sectional shape, which may be useful for manufacture. A circular or oval cross-section can produce muscle tissue in a consistent or even convex shape, thereby producing more and larger pieces of tissue than past systems, as well as tissue which more closely resembles muscle tissue grown in animals.

According to an embodiment, the chamber can be up to 1-2 L in volume, preferably, 250 mL-1.5 L, further preferably 500 mL to 1 L. The use of a chamber with expanding cross-section and three dimensional anchors allows for much larger chambers than seen in the prior art, thereby promoting more efficient muscle tissue production.

According to an embodiment, when the chamber expands in cross-section from each of the ends to a middle point, the middle point is half way between the first end and the second end. Such a design can form a chamber that is substantially symmetric along a horizontal plane at its middle point.

According to an embodiment, the chamber is symmetrical along a central vertical axis between the first end and the second end. This can be useful for efficiency of design and manufacture of the chamber.

According to an embodiment, the first anchor and/or the second anchor are moveable. Optionally, the first anchor and/or the second anchor are movable in a vertical direction and/or rotatable. Such movement(s) can be associated with one or both anchors. For example, one anchor could be rotatable, and one anchor could be moveable in the vertical direction, thereby respectively providing various types of mechanical stimulation to the muscle tissue. Each movement could be powered by separate motors located at or near anchors or connections. Using separate motors could allow for smaller motors, thereby helping to ensure apparatus stays compact overall. Separate motors could also ensure that not all mechanical stimulation ceases should one motor fail. Motors can be electrical and/or battery powered. Making anchors moveable allows for a simple way of providing mechanical stimulation to muscle tissue, helping to promote efficient growth and development of muscle tissue. Using rotational and vertical stimulation allows for putting tension on the muscle tissue in all directions (x, y and z) through various movements, while the anchors ensure that the muscle tissue stays connected throughout the stimulation. In some embodiments, one motor could be used to power both anchors and/or only one anchor could provide both rotational and vertical movements.

According to an embodiment, the first anchor and/or the second anchor are removable from the chamber. These can be through quick release couplings, allowing for simple removal of muscle tissue with anchors upon maturation of the tissue. Such couplings could be, for example, magnetic couplings where a base of the anchor and/or a cap holding the anchor connects to an electromagnet which can be switched on to secure the anchor in place and turned off to remove the anchor/cap with the anchor. Such removable anchors can also help for the cleaning of anchors for reuse (e.g., inserting into an autoclave), or to allow efficient disposal and replacement when anchors are not reusable.

According to an embodiment, the apparatus comprises a heating element. Optionally, the heating element is configured to control a temperature of fluid within the chamber. The heating element can be, for example, heating wires embedded directly in the walls of the chamber to heat up the medium in the chamber through the surface. Optionally, a thermoprobe or other means of sensing the temperature could be used to ensure the temperature inside the chamber and directly surrounding the tissue is precisely controlled. Typically, the temperature for developing muscle tissue would be around 37 degrees C., though could be 32-42 degrees C., or in some cases even outside this range. A further option, in addition or alternative to heating wires could include a jacket around the chamber. Such a jacket could simply be an insulating jacket to help control heat loss, or could surround chamber and have a controlled temperature fluid flow between the jacket and chamber to precisely control the temperature inside of the chamber, thereby controlling conditions for optimal tissue growth and maturation, as well as for the precise temperatures needed for the cooling down and harvesting process.

According to an embodiment, the apparatus further comprises one or more electrodes for providing electrical stimulation. These can be connected, for example, to the first anchor and/or the second anchor for providing the stimulation, though could be connected elsewhere. By directly connecting and/or integrating one or more electrodes into the apparatus, the apparatus is able to provide electrical stimulation to chamber without extra outside parts, thereby promoting cell differentiation and compaction as well as muscle tissue growth.

According to an embodiment, the apparatus could have a manual or automatic control system (or partially automatic and partially manual) to control various aspects of apparatus and the process, such as temperature, pH, flow through the chamber, dissolved oxygen, glucose, etc. Such a control system could connect to sensors, and have various alerts or alarms to warn of conditions which are not ideal or malfunctioning hardware. Such sensors and/or alarms could also indicate the various stages of maturation and alert to maturity markers. Such a system could include various hardware and/or software components, such as one or processors (which could be a special purpose processor for apparatus), memory, a user interface, etc. which could be located at apparatus 10, or remotely (e.g., through a wireless or wired connection). In some instances, one control system could control a number of apparatuses, each apparatus growing tissue.

According to an embodiment, the inlet is located in a shower cap comprising a plurality of outlets configured to flow media into the chamber from an upper end of the chamber at a plurality of points surrounding a central axis of the chamber. Optionally, the shower cap comprises the inlet for receiving the media, a channel for distributing the media around a central axis, and a plurality of outlets connected to the channel for flowing the media out of the channel and into the chamber. Such a design results in outlets being generally located around the circumference or perimeter of the top anchor (below the shower cap) and the eventual tissue. The media flows out of outlets into chamber all around the top anchor and tissue, thereby providing for better flow and distribution of media into and around the tissue and chamber. This leads to better mixing in the chamber as well as better perfusion.

According to a further aspect of the invention, an anchor for a tissue reactor chamber comprises a connector; and a three-dimensional projection extending from the connector. Optionally, this connector can be a connector rod which connects to or through the three-dimensional projection. Further optionally, the three-dimensional projection comprises an outer perimeter extending longitudinally, and a plurality of attachment paths extending from the outer perimeter. Further optionally, the plurality of attachment paths do not align in the longitudinal direction. In other embodiments, the plurality of attachment paths do align in the longitudinal direction. Optionally, the anchor is formed of conductive material and/or food safe biomaterial.

Such a three-dimensional anchor provides a secure point to which muscle tissue can connect when developing in a tissue reactor chamber. The use of a three-dimensional projection with a plurality of attachment paths ensures a large network of points in many directions where the muscle tissue can attach to the anchor, thereby ensuring the connection can withstand stress and tension in all directions (e.g., x, y and z). This allows for mechanical stimulation to help promote efficient tissue growth and maturation as well as quality texture in the muscle tissue produced. Using conductive material allows for electrical stimulation directly through the anchor, and a food-safe biomaterial can help to promote the tissue attachment and growth while ensuring that the material does not otherwise affect the tissue in terms of flavour, look, taste, etc.

According to a further aspect of the invention, a method for growing tissue in a chamber comprises filling a chamber with a suitable scaffolding material comprising cells, the chamber with a first three-dimensional anchor located at a first end and a second three-dimensional anchor located at a second end; and incubating the scaffolding material comprising the cells under conditions suitable for maturation of the cells, whereby the mature cells form tissue connecting to and between the first and second anchors. Such a suitable scaffolding material can optionally be a hydrogel. Optionally, the chamber can be barrel shaped or cylindrical. The filling can be done through an inlet, through the top or any other suitable method.

Such a method is able to produce large pieces of muscle tissue in the chamber between the anchors. In some embodiments, the chamber is substantially barrel shaped. When the chamber barrel shaped, it is especially useful for the production of compacting tissue. The barrel shape is used in this context to refer to a chamber that is expanding in cross-section to have the largest cross-section at a point between the ends. It could be curved, or could come to an apex at the largest cross-section. It was surprisingly discovered that muscle tissue produced with such a process would be either consistent in cross-section between the anchors or even convex, producing more tissue and more closely resembling muscle tissue grown in animals when growing tissue which compacts.

According to an embodiment, the method further comprises providing mechanical stimulation through rotational and/or vertical movement of at least one of the first anchor and the second anchor. Optionally, the method further comprises providing electrical stimulation through one or more electrodes connected to at least one of the first anchor and the second anchor. Such mechanical and/or electrical stimulation can help to more efficiently differentiate as well as elongate and align cells. It can also help to compact the tissue, as well as produce and mature muscle tissue after compaction. In some instances the mechanical and/or electrical stimulation could even be used as a maturity indication (e.g., the tissue has a certain resistance when mature). Due to the use of three-dimensional anchors, greater amounts of mechanical stimulation can be provided without the risk of tearing tissue from anchors, for example, inducing a strain of about 30, preferably 20-100.

According to an embodiment, the step of incubating the scaffolding material comprising the cells under conditions suitable for maturation of the cells, whereby the mature cells form tissue connecting to and between the first and second anchors comprises controlling at least one of the following in the chamber: temperature, pH, dissolved oxygen and glucose; and/or flowing liquid through the chamber. Optionally, liquid can be flowed through the tissue. Such a method uses perfusion and/or circulation around the tissue to promote efficient muscle tissue growth and maturation. Flowing liquid around and/or through the tissue can be done by ensuring there is a constant flow of liquid into the inlet of the chamber and out of the outlet. This can be very slow, for example, 1-2 ml/minute, though could vary, for example, up to 20 mL/minute, with liquid flow rate inside the tissue being lower than around the tissue. Such flow runs growth media around the tissue and into the tissue, promoting healthy growth of muscle tissue in the chamber. The mechanical stimulation can also help to promote the waste media exchange, allowing for the infusion of fresh medium into the tissue. The temperature and/or pH can be controlled with various sensors and/or other components or devices to ensure the correct conditions for promotion of muscle tissue growth and development. Temperature control can be through heating wires directly in chamber wall, an outer jacket (which could include a fluid flow) or other methods.

According to an embodiment, the cells are pluripotent stem cells such as embryonic stem cells or induced pluripotent stem cells. Optionally, the mature cell is a myocyte. In certain embodiments, the mature cells are a mixture of myocytes and adipocytes.

According to an embodiment, the tissue is muscle tissue. Such a method is ideal for growing muscle tissue, particularly that from animals for consumption. In certain embodiments, the muscle tissue further comprises fat tissue.

According to a further aspect, the invention provides for a method of manufacturing engineered tissue products by using the apparatus as described herein. In some embodiments, the engineered tissue products are muscle tissue are intended for research or therapeutic purposes. In certain preferred embodiments, the engineered tissue is muscle tissue intended for dietary consumption by human beings, non-human animals or both. In some embodiments, the engineered muscle tissue products are human food products. In some embodiment, the engineered muscle tissue products are designed to resemble traditional meat products and the cell types are chosen to approximate those found in traditional meat products. Human beings traditionally eat several type of animal muscle tissue. Therefore, in some embodiments, the myocytes are skeletal myocytes or smooth myocytes.

Fat plays a part in the development of the traditional meat flavor during cooking and plays a role in giving meat its characteristic juiciness be enhancing the water-holding capacity of the meat. Accordingly, in one embodiment, the engineered muscle tissue further comprises fat. Therefore, in some embodiments, the muscle tissue further comprises adipose cells.

According to a further aspect of the invention, a chamber for culturing muscle tissue comprises a first end; a second end; and a body with an expanding cross-section from each of the first end and the second end to have a largest cross-section at a point between the first end and the second end. Optionally, the chamber further comprises an inlet and/or an outlet. Further optionally, the body is circular or oval in cross-section. In some embodiments, the chamber has a smooth curvature from each of the ends to the point with the largest cross-section.

Such a chamber can promote the culturing of large amounts of compacting muscle tissue in a desired shape. By having an expanding cross-section from each of the ends, the muscle tissue can compact in a cylindrical or even convex shape, more closely resembling muscle tissue from animals and producing larger pieces of muscle tissue upon compaction.

### BRIEF DESCRIPTION OF THE DRAWINGS

The present invention will be discussed in more detail below, with reference to the attached drawings, in which:

FIG. 1A depicts an apparatus for culturing muscle tissue,

FIG. 1B depicts an exploded view of the apparatus of FIG. 1A

FIG. 2A depicts an embodiment of a chamber for use in the apparatus of FIG. 1A;

FIG. 2B shows a cross-sectional view of the chamber of FIG. 2A, and

FIG. 2C shows a top view of the chamber of FIG. 2A;

FIG. 3A shows a perspective view of an anchor for the apparatus of FIG. 1;

FIG. 3B shows a top view of one plate of the anchor shown in FIG. 3A;

FIG. 4A shows a perspective view of a second embodiment of an apparatus for culturing muscle tissue;

FIG. 4B shows a side view of that apparatus;

FIG. 4C shows an exploded view of the apparatus;

FIG. 5A shows a view of a shower cap, which could be used with the apparatus;

FIG. 5B shows a cross-sectional view of the shower cap of FIG. 5A;

FIG. 6A shows a perspective view of an alternative anchor;

FIG. 6B shows a side view of the anchor of FIG. 6A;

FIG. 6C shows a top view of the anchor of FIG. 6A;

FIG. 6D shows a schematic depiction of the anchor in a relaxed state;

FIG. 6E shows a schematic depiction of the anchor in a stretched state;

FIG. 6F shows the anchor of FIGS. 6A-6E in use in a chamber;

FIG. 6G shows a top view of the chamber of FIG. 6F;

FIG. 7A shows a perspective view of a building block of the alternative anchor;

FIG. 7B shows a side view of the building block; and

FIG. 7C shows a top view of the building block.

### DESCRIPTION

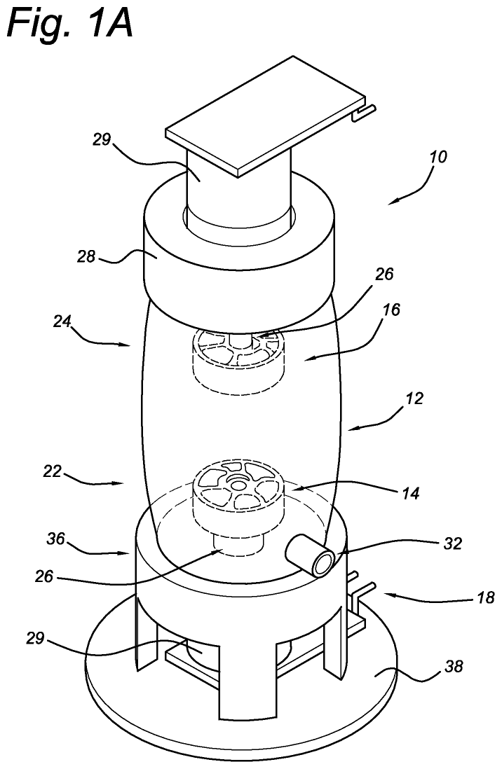

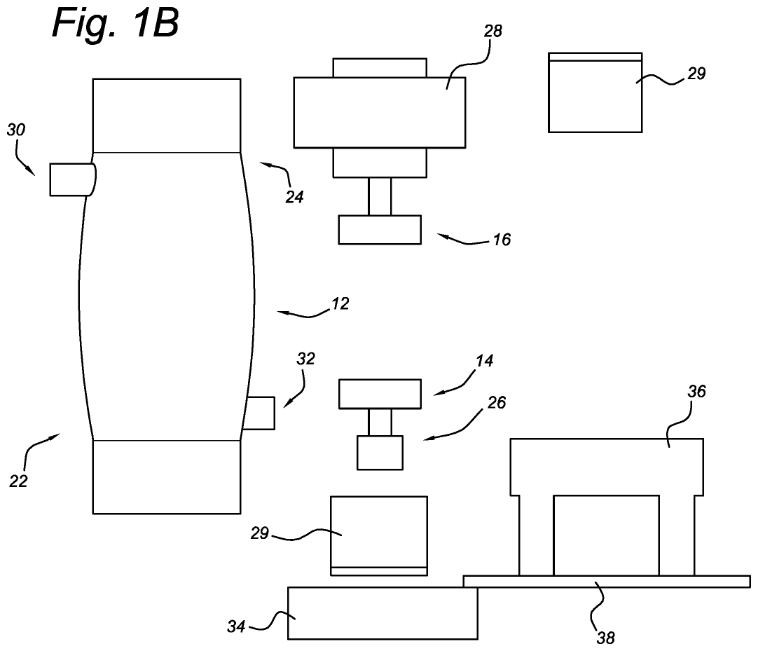

FIG. 1A depicts an apparatus 10 for culturing muscle tissue, and FIG. 1B depicts an exploded view of the apparatus. Apparatus 10 includes chamber 12, anchors 14, 16, base 18, cap 28. Anchors 14, 16 each include a connector rod 26. Chamber 12 and anchors 14, 16 are aligned around vertical axis A. Chamber 12 includes inlet 30, outlet 32 and cavity 33. Base 18 includes fan 34, holder 36 and metal sink 38.

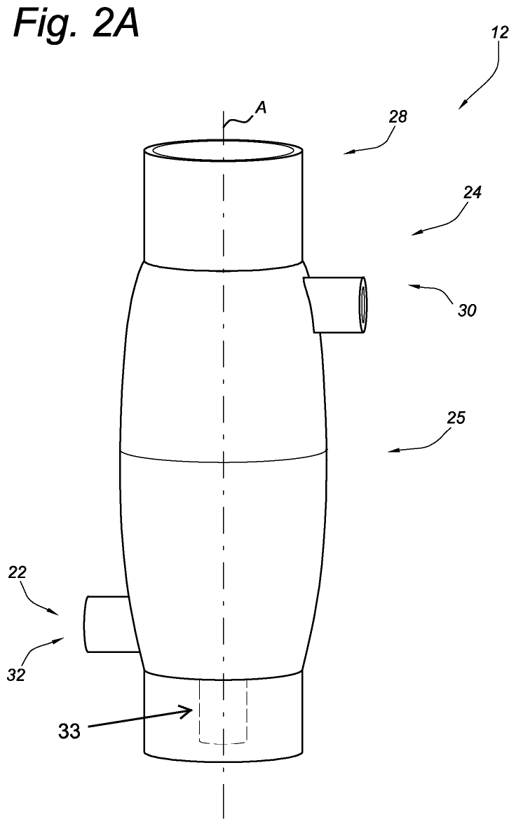

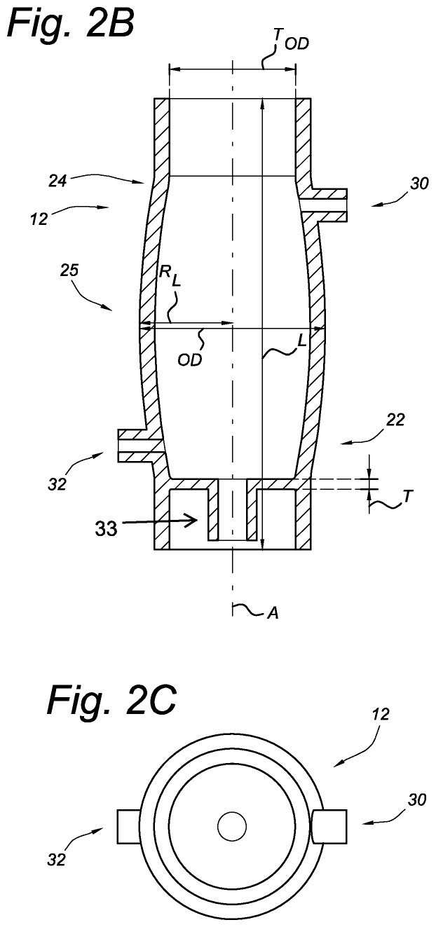

Base 18 acts to support and stabilize chamber 12, anchors 14, 16 and other supporting parts not shown (motor(s), electrode(s), pump(s), etc.). In the embodiment shown, the chamber is supported from base 18, extending longitudinally from holder 36 at first end 22 to cap 28 on second end 24 of chamber 12. Other embodiments, for example, chambers which are not supported from the ground (e.g., suspended) could have different support configurations, changing and/or eliminating one or more of base 18, and cap 28. Cap 28 and connections with chamber 12 can be better seen in FIG. 2A, and the exploded view in FIG. 1B.

Each of anchors 14, 16 secure to electromagnets 29 for positioning within chamber 12. Anchor 14 connector 26 is positioned within cavity 33, which can be a blind hole (or a through cavity in some cases) in proximity to electromagnet 29 at holder 36. Metal sink 38 acts as a heat sink, and fan 34 helps to blow the heat away so it does not affect the chamber 12 temperature or the muscle tissue culturing. Holder 36 helps to position anchors 14, 16 at a sufficient distance from electromagnet 29 so that the magnetic field generated does not affect the muscle tissue grown. Cap 28 can connect to chamber 12 at second end 24, with anchor 16 connecting to electromagnet 29 through cap 28. Cap 28 can be configured to allow anchor 16 to move freely. Cap 28 can also have a fan inside, which is not shown in the Figures. In some embodiments, cap 28 could be formed integral with chamber 12. Cap 28 could also be used to house other parts of apparatus 10, such as motors for mechanical stimulation, electrodes, sensors, alarms, etc. In some embodiments, cap 28 could be hollow to allow for easy removal of muscle tissue with anchors through one end of chamber 12 and cap 28. Alternatively, the cap 28 could be removable with the anchor 16 and tissue at the time of harvest.

Chamber 12, which is depicted in more detail in FIGS. 2A-2B, extends from a first end 22 to a second end 24 along vertical axis A and connects to holder 36 of base 18 at first end 22 and second end 24 to cap 28. Chamber 12 has an outer wall that expands in cross-section from each of the first end 22 and the second end 24 to have a largest cross-section at a point between the first end 22 and the second end 24. This point should be about half way between the first anchor 14 and the second anchor 16, which will often correspond to the half way point between the first end 22 and second end 24, though this could differ depending on anchor 14, 16 placement within chamber 12. The shape is generally curved, for example, barrel or oval shape between the connecting ends 22, 24, though does not have to be curved (e.g., could be conical or slanting from both ends). Chamber is typically formed of a transparent material, such as glass or plastic to enable viewing through the chamber. In some embodiments, chamber could have only limited or no viewing windows (e.g., made of stainless steel), which may help with maintaining temperature, consequently leading to energy savings. Chamber can also include various heating elements and/or temperature control devices, such as heating wires and a thermoprobe, which will be described in more detail.

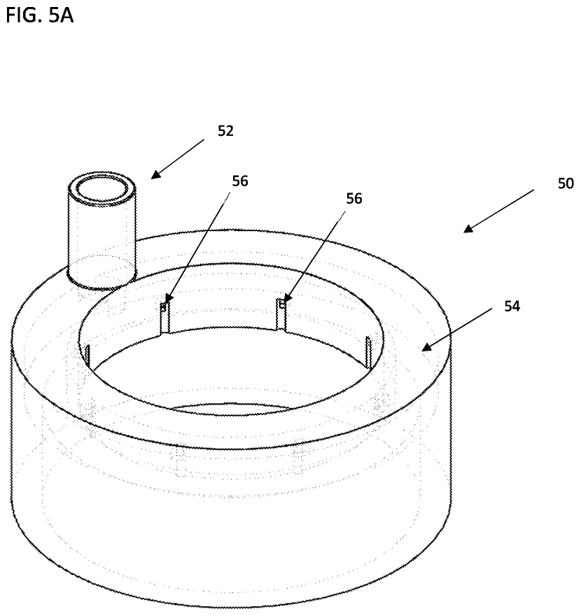

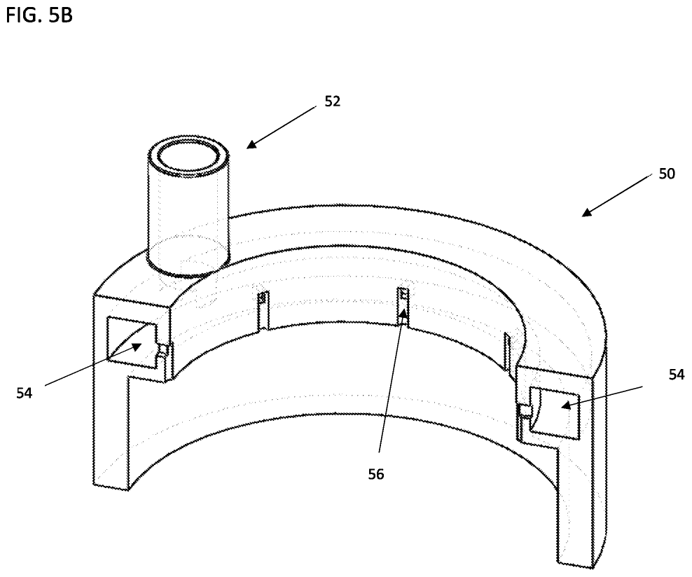

Chamber 12 includes an inlet 30 and outlet 32. Inlet 30 and outlet 32 allow for controlling the flow of fluid into and out of chamber 12. Inlet 30 and outlet 32 placement are shown for example purposes only and could be located at different points in the chamber. Some embodiments could have more than one inlet and/or outlet, for example, a large inlet and outlet used only for initial filling of chamber 12 and draining at harvest with a smaller inlet and outlet for flow during tissue growth processes. An alternative inlet is depicted in FIGS. 5A-5B.

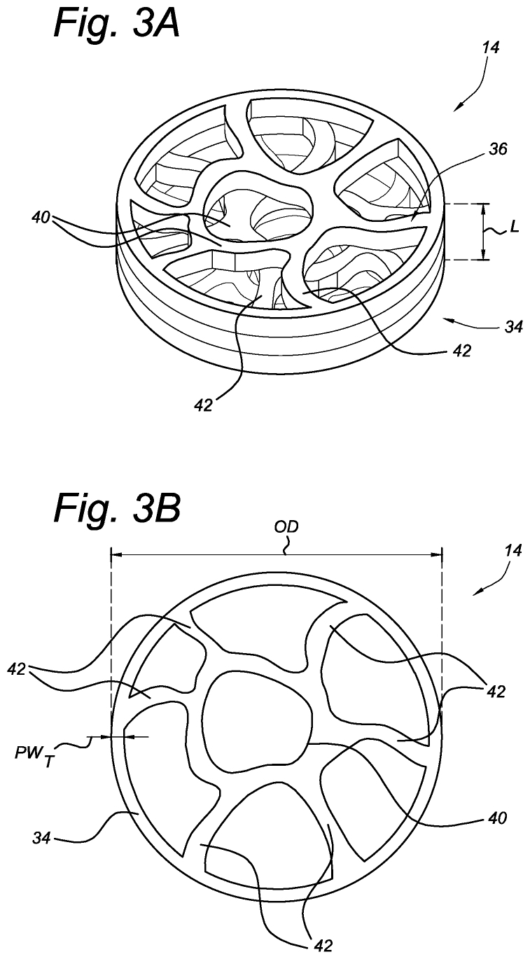

Anchors 14, 16 are biomimetic anchors, which are each located within chamber 12 at opposite ends. The specific design of anchors 14, 16 will be discussed in more detail in relation to FIGS. 3A-3B. Anchors 14, 16 are formed of conductive material, for example metals such as Titanium or stainless steel and/or carbon based materials such as graphite and graphene. At least one of anchors 14, 16 are connected to an electrode or other device to provide electrical stimulation to the chamber through the anchor(s). The electrode or other device could be connected to the anchor(s) through the connecting rod(s) 26. In some embodiments, electrodes are integrated into the connector rods 26 or anchor 14, 16 to provide electrical stimulation without the need for separate electrodes and attachments. Anchor 14 can rotate, with power provided by a connected motor (typically located at an end of connector rod 26 in cap 28). Anchor 16 can move up and down along the vertical axis A, with power provided by a separate motor (also not shown, but can be located within cap 28). In some embodiments, anchor 14 could move vertically and/or anchor 16 could rotate. Additionally, some embodiments could power both anchor 14, 16 movements with one motor, though having separate motors can reduce the risk of unintentionally ending all stimulation or movement due to motor failure and allow for smaller motors within apparatus 10. In further embodiments, only one of anchors 14, 16 could perform both the longitudinal and rotational movement. Motors can be battery powered and/or electric.

Apparatus 10 functions to grow muscle tissue in chamber 12 between anchors 14, 16. Anchors 14, 16 are placed within chamber, with electromagnets 29 securing anchors 14, 16 in place. Then chamber 12 is filled with a hydrogel (or other suitable scaffolding material) through the top and/or inlet. The chamber interior is typically filled fully with the hydrogel with suspended cells.

The cells used could be a number of different cells. In one embodiment, the cells are preferably self-renewing cells such as pluripotent stem cells, or any type of muscle cell. Pluripotent stem cell include embryonic stem cells or induced pluripotent stem cells (iPSC) that maintain the capacity to self-renew in the undifferentiated state, or alternately differentiate to any tissue lineage. In certain embodiments, the cells originate from an animal species intended for dietary consumption, including livestock, poultry and game. In a preferred embodiment, the cells are from bovine or porcine origin. In certain aspects, the cells originate from species intended for research or therapeutic purposes such as humans, primates and rodents including rats and mice.

Chamber conditions are precisely controlled to promote the differentiation and subsequent compaction and maturation of the cells. Typically, the chamber would be kept at about 37 degree C., and pH, dissolved oxygen and/or glucose would be controlled. Culture conditions suitable for the differentiation of pluripotent cells and subsequent maturation can be maintained within the chamber. Cells would then begin differentiating, resulting in tissue compaction. Compaction would typically take about 1-14 days, though this depends on the type of cells, chamber conditions, hydrogel, etc. The cells compact to around half their original volume and begin to attach themselves to anchors 14, 16 at many points around the three-dimensional pathways through anchors 14, 16. During compaction, cells interact with the hydrogels, pulling fibers and expelling water to compact together and form muscle tissue attaching to and between anchors 14, 16. Compact cells then mature into muscle tissue, typically in about 12-18 days.

Electrical stimulation through the connection of at least one electrode to at least one of the anchors 14, 16 can begin as soon as the hydrogel fills the chamber 12, even before compaction as it can help with cell differentiation, stimulating cells to align, fuse and connect. Such electrical stimulation can help to promote compaction with the cells and promote muscle tissue growth and maturation after compaction.

Mechanical stimulation through movement of one or more anchors 14, 16 can begin after compaction, when muscle tissue has attached to each of anchors 14, 16. Sometimes a waiting period after compaction is desirable, for example, two days, to ensure there is a strong connection to anchors 14, 16 and that the tissue has developed sufficient integrity and strength to endure the movements before starting mechanical stimulation. The mechanical stimulation can include rotational movement through anchor 14 and/or vertical movement through anchor 16 and/or anchor 14. Movements must be tailored relative to the size, and typically should not result in strain more than 0.3, preferably no more than 0.2. The three-dimensional anchors 14, 16 with a plurality of attachment paths allow for larger mechanical stimulation due to the stronger attachment of the tissue in x, y and z directions to the anchors 14, 16.

Chamber 12 also promotes tissue growth by perfusion (liquid inside the tissue) and perifusion (liquid flowing around the tissue). Specifically, fresh liquid (e.g., water) is flowed through chamber 12 inlet 30 and out of outlet 32 at a constant rate. In some embodiments, the flow could be through separate inlets and outlets designed specifically for perifusion (e.g., sides or ends of the chamber at places where the tissue will reside to ensure flow all around the tissue). This flow around the tissue is typically a rate of around 10 mL-100 mL per minute, with perfusion flow at a very low rate, for example, 0.5-10 mL per minute. Such flow runs culture media around the tissue and into the tissue, promoting healthy growth of muscle tissue in chamber 12. The mechanical stimulation, for example, rotation, can also help to promote perfusion, allowing for the infusion of new medium into the tissue, and expelling exhausted media out of the hydrogel. The temperature, pH, dissolved oxygen and glucose are typically controlled with various sensors and/or other components to ensure the correct conditions for promotion of muscle tissue growth and development. The temperature control can be through heating wires directly in chamber 12 wall, an outer jacket (which could include a fluid flow) or other methods.

After about 12-18 days, muscle tissue develops into a mature shape connected to and extending between anchors 14, 16. Once the muscle tissue has reached a point of maturity, the tissue can be removed from the chamber 12 in a harvesting operation. Maturity can be determined in a number of ways, for example, testing of mechanical strength, resistivity, color, density, biomarkers such as myoblast determination protein, etc. Devices and/or sensors which form a part of apparatus 10 (e.g., mechanical or electrical stimulation components) could be used for testing and recognizing maturity, or outside sensors can be used. Maturity marker testing can be done manually, or automatically, for example, through a control system.

Chamber 12 could then be flushed with water, or simply drained through outlet 32 and can be sterilized, for example, in an autoclave. Electromagnets 29 can be deactivated to release anchor 14 from base 18. Other embodiments could be released and/or decoupled through a plug, button or other quick release mechanism either in place of or in addition to electromagnet 19. Anchor 16 (and all or part of cap 28) also has a quick release coupling, deactivating electromagnet 29, which allows anchor 16 to be released from chamber 12. Anchors 14, 16 with muscle tissue are extracted from chamber 12 vertically upwards along vertical axis A through second end 24 of chamber 12. This can be manually or through a machine, arm, or other device. The grown tissue connected at one end to anchor 16 and at the other end to anchor 14 moves with anchor 16 (also bringing anchor 14) out of the top of chamber 12. Once removed from chamber 12, tissue can be removed from anchors 14, 16, e.g., by cutting.

Apparatus 10, through the use of a specific chamber 12 shape and three-dimensional, biomimetic anchors 14, 16 is able to efficiently produce large, three-dimensional muscle tissue. Past systems for growing muscle tissue in a chamber typically either grew very small amounts of tissue (e.g., 500 microliters to 2 milliliters) or two-dimensional tissue. In some systems where three dimensional tissue was grown, it would typically connect to anchor points and have a very thin connection in the middle of anchor points with a concave or hourglass shape. The shape of chamber 12 allows for the growth of muscle tissue with a substantially uniform cross-section, or even a convex shape between anchors 14, 16, resulting in more tissue produced and a more desirable muscle tissue shape which more closely resembles meat from animal. This shape is typically more desirable for use (being larger) and for consumer preferences.

The use of three-dimensional biomimetric anchors 14, 16 promotes stronger connections of muscle tissue to anchors, thereby allowing for efficient production of larger quantities of muscle tissue, as well as increased mechanical stimulation resulting in muscle tissue with increased quality in terms of texture, taste and visual appearance. The three-dimensional biomimetric anchors 14, 16 also allow for a large amount of connection points for muscle tissue with anchors 14, 16, resulting in decreased risk of the muscle pulling away from anchors 14, 16, even during relatively strong mechanical stimulation. The ability to easily remove anchors 14, 16 with quick coupling mechanisms also ensures a simple and efficient harvest operation. Additionally, the use of metallic or other autoclavable material for anchors 14, 16, and possibly chamber 12 ensures a relatively simple cleaning process and allows for reuse of apparatus. In some embodiments, anchors 14, 16 could be non-reusable parts and simply discarded after muscle tissue is removed during harvesting. The use of electromagnets 29 allows for easy and quick coupling and uncoupling, and holder 36 and cap 28 help to ensure the magnetic field does not affect the tissue growth. The use of fans 34 and metal sink 38 also ensure that the heat generated does not affect the growing conditions within chamber 12.

FIG. 2A depicts an embodiment of a chamber 12 for use in apparatus 10; FIG. 2B shows a cross-sectional view of the chamber 12; and FIG. 2C shows a top view of the chamber 12. Chamber 12 includes first end 22, second end 24, largest cross-sectional point 25, inlet 30, outlet 32 and cavity 33. Cavity 33 at first end 22 can be a blind hole and/or shelf for receiving and properly positioning anchor 14; and second end 24 is generally cylindrical in shape and open for cap 28 to cover or connect to and through.

Chamber dimensions include length L of 70-80 mm, radius R_{L} at largest cross-section of about 15.5 mm, radius R_{FE} at first end of 5 mm; top opening diameter TOD of 25 mm; outer diameter OD at thickest part of about 36 mm, and an outer wall thickness of 3 mm. These are example dimensions for a chamber of 250 mL, and dimensions would vary depending on the size of chamber required, the muscle tissue being produced, the material used for chamber, conditions for production of muscle, etc. Such a configuration could also be used for chambers from 25 mL up to 1-2 L or even more, for example 500 mL to 1 L. This allows form much larger chambers, and consequently larger portions of cultured tissue than prior art chambers.

Chamber 12 can be formed of glass, though could be a metal such as stainless steel, or other suitable materials. Chamber can be formed by additive manufacturing, casting and milling or other suitable manufacturing processes.

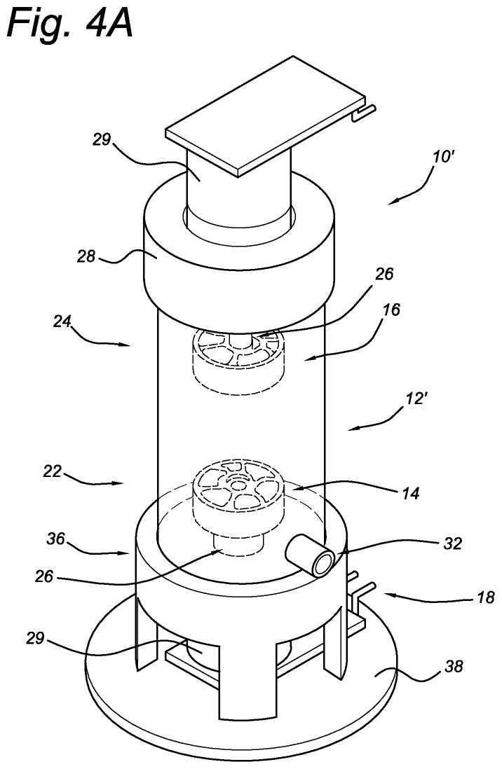

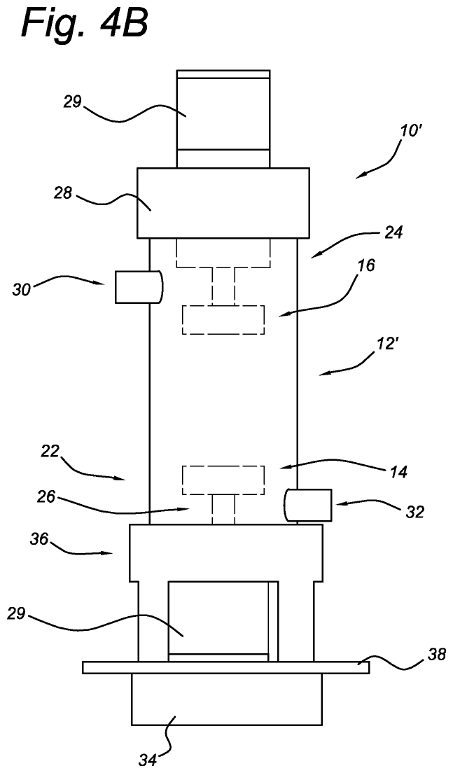

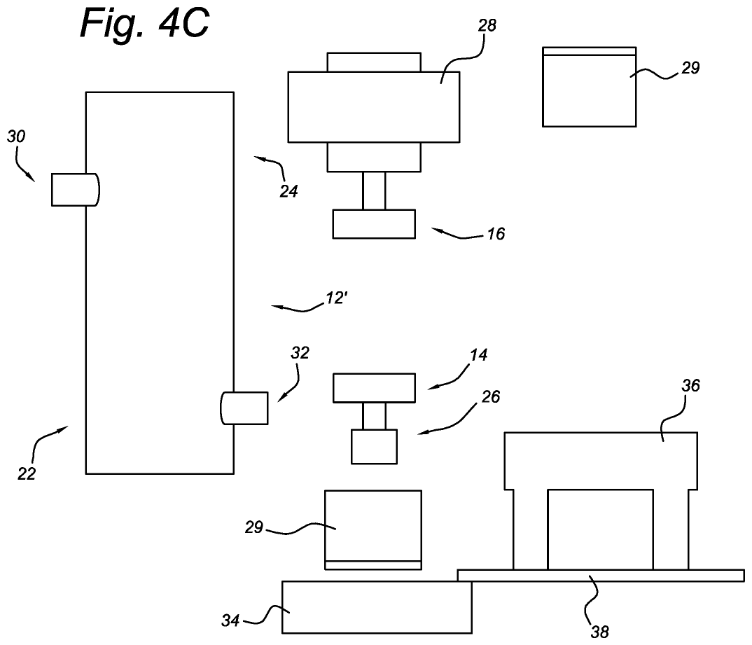

Chamber 12 is symmetric around its vertical axis, and shaped to have an expanding cross-section from each of its ends 22, 24. The cross-section is largest at middle point 25, which is generally around a mid-point of chamber 12 between ends 22, 24, but does not have to be. In this embodiment, cross-sections are circular though they could be different shapes (e.g., oval) in other embodiments. The walls of chamber 12 shown in FIGS. 2A-2C are curved, though other embodiments could have less (or more curvature), including embodiments with little to no curvature as that shown in FIGS. 4A-4C.

The design of chamber 12 with an expanding cross-section from each of the ends 22, 24 allows for production of muscle tissue with consistent shape between anchors. Prior art chambers typically had straight or parallel sides (e.g., rectangular or cylindrical), which resulted in growth of muscle tissue that had a larger cross-section at the ends, and a smaller cross-section in the middle, or concave sides with an hourglass shape in the tissue produced. It was surprisingly discovered that designing chamber 12 to have an expanding cross-section from each of the ends with a largest cross-section at a middle point resulted in muscle tissue that was substantially uniform or even convex between anchors 14, 16, thereby resulting in more muscle tissue growth (larger volume), and overall more efficient production. The shape was also more desirable for usage and for consumer preferences.

In some embodiments, the curvature of chamber 12 is different, for example, the chamber could expand even further to have a largest cross-section much larger than ends and anchors 14, 16. Such a configuration could be used to form muscle tissue with a convex shape, which more closely aligns with that from animals—closely mimicking muscle tissue which consumers expect from non-lab grown muscle tissue.

Chamber 12 wall can include one or more heating wires directly in the walls of chamber 12 to precisely regulate the temperature inside chamber 12. Alternately or additionally, chamber could include an outer sleeve to help regulate temperature. Some embodiments could even include a fluid flow between the outer sleeve or jacket and the chamber 12 to help control temperature in an efficient and economical way. The growth of muscle tissue is a very temperature sensitive process, and certain parts of the procedure, such as the cooling down must be done very precisely to ensure that the texture of the muscle tissue does not degrade during the process. Such heating wires or other temperature regulators built directly into apparatus 10 can ensure that the temperature is accurately and precisely controlled.

FIG. 3A shows a perspective view of a three-dimensional anchor 14; and FIG. 3B shows a cross-sectional view of anchor 14. While anchor 14 is referenced, this could also be anchor 16. Anchors 14, 16 would typically have the same configuration, though could be different in some embodiments. Anchor 14 is in a biomimetic configuration, resembling a tendon from an animal. Example dimensions can include an outer diameter OD of 25 mm, length L of 2-10 mm, and a perimeter wall thickness PWT of 1 mm. Anchor 14 dimensions, particularly length, can vary depending on the volume of chamber and attachment strength required. The anchor 14 shown would be suitable for use with the apparatus of FIGS. 1A-1B, 4A-4C and/or the chamber of FIGS. 2A-2C.

Anchor 14 is a biomimetic shape, modelled after a projection of a tendon, and having a three-dimensional shape to which tissue can attach. Anchor 14 includes perimeter 34 and a plurality of attachment paths 36. Attachment paths 36 form various three-dimensional pathways throughout the interior of perimeter 34. In the embodiment shown, perimeter 34 is cylindrical, but could be another shape in other embodiments. Perimeter 34 typically has a solid outer wall extending longitudinally, with the paths 36 forming winding interconnected pores through the longitudinal direction within perimeter 34. Attachment paths 36 do not typically extend the length of perimeter, and instead different paths 36 start at different points along the inner circumference of perimeter 34, and do not generally align in the vertical direction, though in some embodiments, they could align. The embodiment shown has a sort of wheel and spoke pattern, with an open inner hub 40 and spokes 42 extending from the inner hub 40 to the outer perimeter 34. Such a configuration can be useful for a connector rod 26 (see FIG. 1A) to extend into anchor 14 inside of inner hub 40, thereby securely connecting to anchor 14. In some embodiments, the same pattern shown in the cross-section of FIG. 3B could be simply repeated and rotated along the vertical axis of anchor 14 inside perimeter 34 to form the plurality of attachment paths 36 connecting at various three-dimensional points with each other and with the perimeter 34.

Anchor 14 can be formed of a conductive material, for example metals or alloys, e.g., Titanium. Anchor 14 can be formed by casting, machining, or other methods depending on the specific design, materials and size. Using a conductive material allows for connection to one or more electrodes to provide direct electrical stimulation to chamber 12 and muscle tissue for more efficient compaction and growth.

Cross-sectional view of anchor 14 shown in FIG. 3B also shows example attachment points 38 for muscle tissue to pathways 36. As can be seen from this cross-sectional view, the plurality of pathways 36 result in many points 38 for the muscle tissue to connect or attach to anchors 14, both along the perimeter 34 and along all sides of the pathways 36. The three-dimensional configuration allows for different attachment points 38 in different planes, thereby giving the muscle tissue stronger connections to anchors 14, 16 in the x, y and z directions.

As mentioned above, mechanical stimulation helps in the efficient growth and development of muscle tissue. The three-dimensional configuration of anchor 14 with a large number of attachment points 38 in all directions allows for mechanical stimulation to put tension on the muscle tissue in all directions (x, y and z) through various movements, while maintaining a reduced risk of muscle tissue detaching from anchor 14. With such a configuration, mechanical stimulation can be performed (e.g., rotational and/or vertical movements) which would result in strain of up to 0.2-0.3, both in the longitudinal and rotational direction. In the chambers shown, this would be about 1 mm of movement, though the movement amounts would vary depending on the muscle tissue being grown, anchor design, size of chamber, etc. Past systems could only perform mild mechanical stimulation due to the risk of detachment of muscle tissue from anchors. By using the three-dimensional biomimetic anchors 14, 16 where muscle tissue wraps itself around and through to attach at a large number of points on all sides of attachment paths 36 and inside of perimeter 34 of anchor 14, apparatus 10 is able to produce larger volumes of muscle tissue more efficiently. Anchors 14, 16 are also able to provide a large number of attachment points 38 in a compact volume, thereby allowing for easy removal from one end of chamber 12 (through top opening) when muscle tissue has matured.

FIG. 4A shows a perspective view of a second embodiment of apparatus 10′ for culturing muscle tissue; FIG. 4B shows a side view of that apparatus 10′; and FIG. 4C shows an exploded view of apparatus 10′. Similar parts are labeled similarly to those in FIGS. 1A-3B.

Apparatus 10′ includes chamber 12′ (with inlet 30 and outlet 32), anchors 14, 16, connector rods 26, cap 28, electromagnets 29, fan 34, holder 36 and metal sink 38.

Apparatus 10′ works in the same manner as apparatus 10 described in relation to FIGS. 1-3B. However, in this embodiment, chamber 12′ is generally cylindrical from the first end to the second end. This can be useful for culturing muscle tissue which compacts very little during maturation, allowing for growing a generally cylindrical muscle tissue between anchors 14, 16.

Each of anchors 14, 16 is metallic and secures to electromagnets 29 for positioning within chamber 12′ for culturing muscle tissue. Metal sink 38 acts as a heat sink, and fan 34 helps to blow the heat away so it does not affect the chamber 12′ temperature or the muscle tissue culturing. Cap 28 can also have a fan inside, which is not shown in the Figures. Holder 36 helps to position anchor 14 at a sufficient distance from electromagnet 29 so that the magnetic field generated does not affect the muscle tissue. Cap 28 can perform a similar function.

The electromagnetic coupling of anchors 14, 16 allows for secure positioning of anchors 14, 16 and therefore tissue within chamber 12′, and for easy and quick uncoupling when it is time to harvest the tissue. For culturing tissue, anchor 14 is placed on holder 36, with cap 28 holding anchor 16 placed on top of chamber 12′. Electromagnets 29 are then activated, and a magnetic field is produced, which holds each of anchor 14 and 16 in place, respectively. When the tissue has been cultivated (as described above), electromagnets 29 are deactivated and cap 28 with anchor 16 can be moved vertically upwards out of chamber 12′. This can be manually or through a machine, arm, or other device. The grown tissue connected at one end to anchor 16 and at the other end to anchor 14 moves with anchor 16 (also bringing anchor 14) out of the top of chamber 12′. Once removed, tissue can be removed from anchors 14, 16, e.g., by cutting.

Chamber 12′ with anchors 14, 16 and electromagnetic couplings provides a system where relatively large amounts of muscle tissue can be grown and easily removed when maturation has taken place. The use of electromagnets 29 allows for easy and quick coupling and uncoupling, and holder 36 and cap 28 help to ensure the magnetic field does not affect the tissue growth. The use of fans 34 and metal sink 38 also ensure that the heat generated does not affect the growing conditions within chamber 12′. Cylindrical chamber 12′ is particularly useful when growing tissue which experiences little to no compaction.

FIG. 5A shows a view of a shower cap 50, which could be used with apparatus 10, 10′ as an alternative way to introduce liquid and/or media into chamber 12, 12′. FIG. 5B shows a cross-sectional view of shower cap 50.

Shower cap 50 includes inlet 52, channel 54 and outlets 56. Generally the shower cap would connect to chamber 12, 12′ at the upper end either inside of or in place of cap 28. Anchor 16 would fit through the center of shower cap 50 and could be moveable up and down with respect to shower cap, as described above.

Media would enter into shower cap 50 through inlet 52 and flow into annular channel 54. Outlets 56, which could be holes, microtubes or other outlet configurations, are equally spaced around channel 54 and around a central axis of the chamber. Thus, the outlets 56 are generally located around the circumference or perimeter of the anchor below and the eventual tissue. The media flows out of outlets 56 into chamber 12, 12′.

The equidistant spacing and plurality of outlets 56 located all around the shower cap 50 provide an inflow of media all around top anchor 16 and tissue, thereby providing for better flow and distribution of media all around the tissue and chamber. This leads to better mixing in the chamber as well as better perfusion. Typically shower cap 50 would eliminate side inlet 30 shown in chambers 12, 12′ being used in place of that media inlet. However, in some embodiments, shower cap 50 could be used in addition to inlet 30.

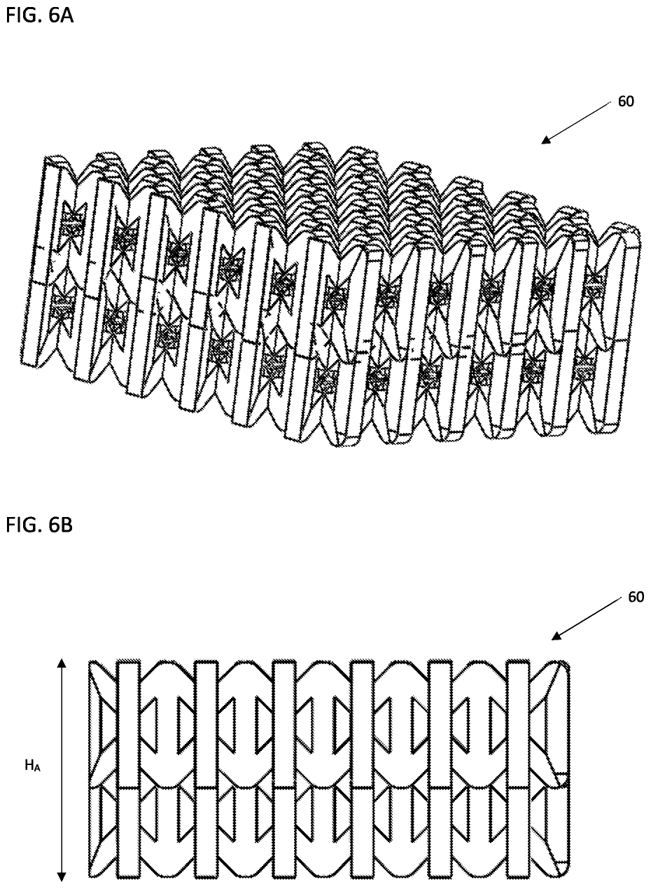

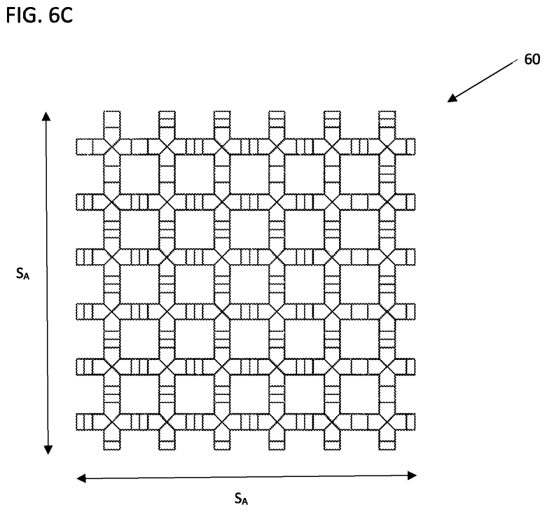

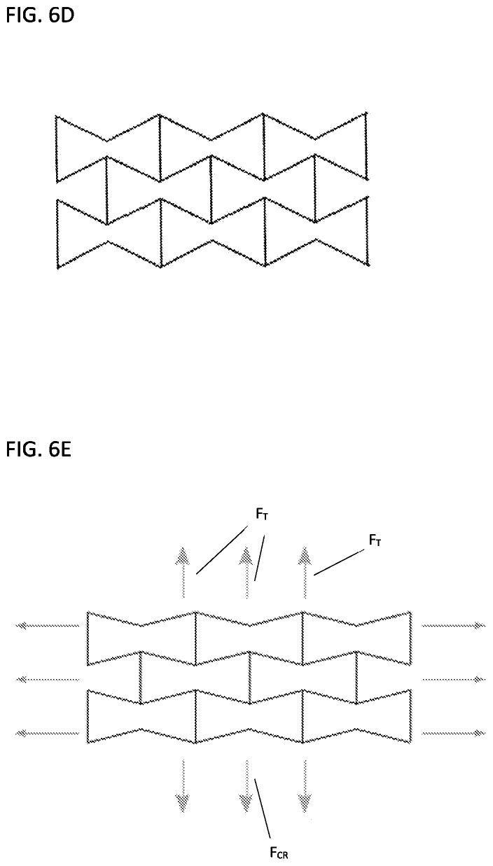

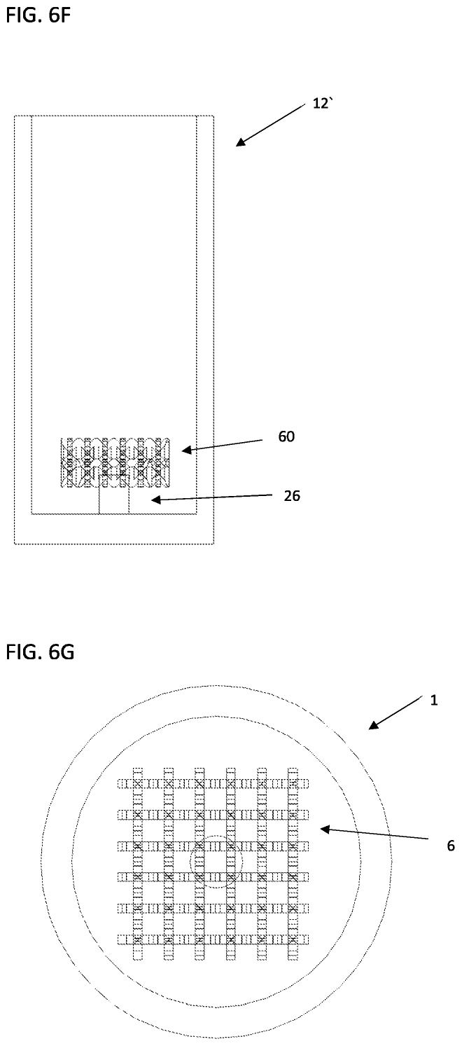

FIG. 6A shows a perspective view of an alternative anchor 60; FIG. 6B shows a side view of anchor 60; FIG. 6C shows a top view of anchor 60. Anchor 60 is an alternative to anchors 14, 16 depicted and described above. Anchors 60 can be formed of a metallic material, or other suitable materials.

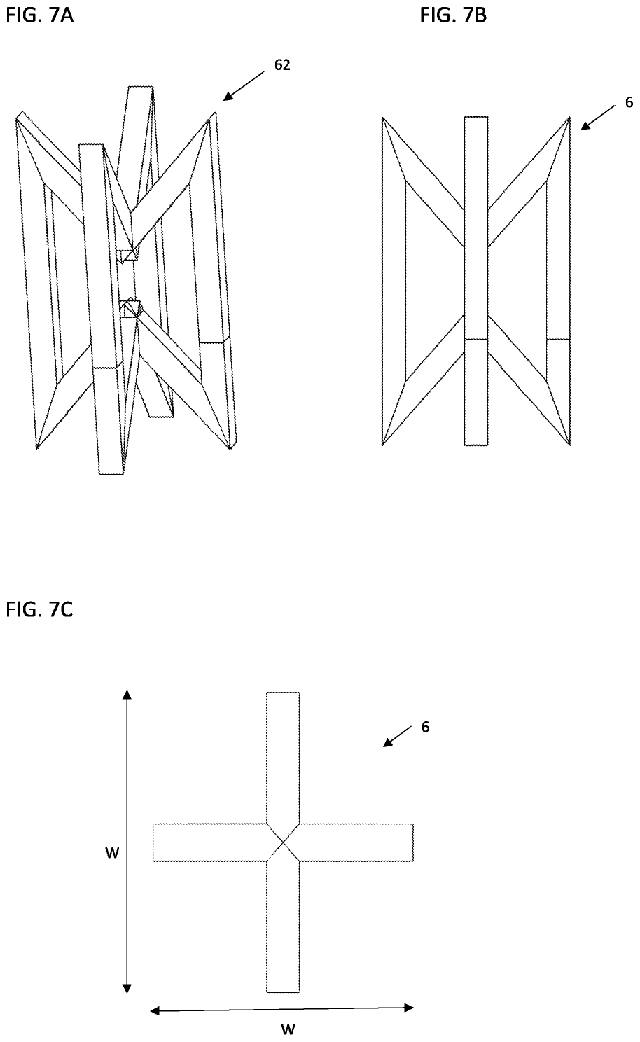

Anchor 60 is an auxetic design, with the building blocks 62 shown in FIGS. 7A, 7B and 7C. The building blocks 62 shown are butterfly shaped hexagon prisms connected at a center point, which can deform into square or rectangular shapes when an outward force is applied. The building blocks 62 are repeated in the x-direction, where left vertical bar of the right block also functions as the right vertical bar of the left block. In the y-direction, the blocks 62 are simply stacked. In the embodiment shown, anchor 60 extends two building blocks 62 in the vertical direction, and is a square shape, extending six building blocks 62 on each side, as seen in FIGS. 6B and 6c. Other embodiments could have a different shape, for example, circular or another polygonal shape and/or a different configuration.

Example dimensions can be HA of about 4.9 mm; WBB of about 4.0 mm; and a side length S_{A} of about 10.87 mm. These are example dimensions only, and can vary depending on chamber and tissue requirements.

FIGS. 6D and 6E show schematic depictions of how anchor 60 behaves, with FIG. 6D showing anchor 60 in a relaxed state; and FIG. 6E shows anchor 60 in a stretched state. The auxetic design of anchor 60 means that when stretched or elongated in one direction, the anchor becomes thicker perpendicular to the applied force. Thus, the relaxed state, as shown in FIG. 6D is more compact than when forces, such as tissue growth and/or mechanical stimulation put forces on anchor. Force Fr could represent the force of the tissue pulling anchor 60 upward in a vertical direction, with force FcR representing the force of the connector rod holding anchor 60 in place within the chamber. These two forces acting on anchor 60 result in the horizontal forces seen elongating the anchor in that direction, perpendicular to the forces applied.

FIG. 6F shows anchor 60 in use in a chamber 12′; and FIG. 6G shows a top view of anchor 60 in use in chamber 12′. Chamber 12′ is a schematic depiction and does not include all parts which chamber 12′ would typically have (as is shown in FIGS. 1A2C and 4A-4C). Anchor 60 is shown at the lower end of chamber 12′, connected through connecting rod 60, similar to anchors 14 shown and described in the previous figures. Chamber 12′ would also include a top anchor 60, extending from a connecting rod from the upper end of the chamber 12′. Tissue would connect and grow between the upper and lower anchors 60.

Anchor 60, by forming from a plurality of building blocks 62 in an auxetic design, provides a large amount of places for tissue to wrap around and to which to connect. The use of relatively small building blocks providing small pores has been shown to result in a positive influence in the attachment of the tissue to anchor 60. The auxetic design allows for the use of these small building blocks 62 in a repeated manner to build up anchor 60 to any size required. Due to the specific design of the building blocks 62 and anchor 60, the attachment points can be located on a number of sides, both in the interior and on the outside of the anchor 60. This results in very strong connections between the anchor 60 and tissue, allowing for the growth of larger muscle tissue and for more mechanical stimulation, resulting in more efficient growth and muscle tissue quality in terms of texture, appearance and taste. Additionally, the auxetic design provides for additional strength, even when subject to the mechanical stimulation loads, with forces in one direction resulting in elongation in another. The use of a metallic or conductive material to form anchor 60 allows for the direct connection and/or integration of one or more electrodes into the apparatus to provide electrical stimulation to chamber, to promote cell differentiation and tissue compaction as well as muscle tissue growth systems and components shown and discussed in relation to FIGS. 1A-2C can be used with apparatus 10′ and chamber 12′ of FIGS. 4A-4C, and vice versa. Systems and components shown and described in FIGS. 5A-7C could also be used with any of the chambers and apparatuses shown.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

1. An apparatus for culturing muscle tissue, the apparatus comprising:
a chamber extending from a first end to a second end;
a first three-dimensional anchor located within the chamber at the first end;
a second three-dimensional anchor located within the chamber at the second end;
an inlet; and
an outlet,
wherein each three-dimensional anchor is an auxetic design.

2. The apparatus of claim 1, wherein the chamber has an expanding cross-section from each of the first end and the second end to a point between the first end and the second end.

3. (canceled)

4. The apparatus of claim 1, wherein the chamber is cylindrical.

5. The apparatus of any of claim 1, wherein each three-dimensional anchor comprises an outer perimeter and a plurality of attachment paths extending from the outer perimeter.

6. (canceled)

7. (canceled)

8. (canceled)

9. The apparatus of claim 1, wherein each three-dimensional anchor is formed of a plurality of auxetic building blocks connected together.

10. The apparatus of claim 9, wherein the anchor is a square or rectangular configuration with a plurality of building blocks connected together in at least two horizontal planes connected to each other vertically.

11. The apparatus of claim 1, wherein the chamber cross-section is circular, oval, rectangular or triangular and/or wherein the chamber is symmetrical along a central vertical axis between the first end and the second end.

12. (canceled)

13. The apparatus of claim 1, wherein the first anchor and/or the second anchor are moveable and/or rotatable.

14. (canceled)

15. The apparatus of claim 1, where the first anchor and/or the second anchor are removable from the chamber.

16. The apparatus of claim 1, and further comprising a heating element and/or one or more electrodes for providing electrical stimulation.

17. The apparatus of claim 16, wherein the heating element is configured to control a temperature of fluid within the chamber.

18. (canceled)

19. (canceled)

20. The apparatus of claim 1, wherein the inlet is located in a shower cap comprising a plurality of outlets configured to flow media into the chamber from an upper end of the chamber at a plurality of points surrounding a central axis of the chamber.

21. (canceled)

22. An anchor for a tissue reactor chamber, the anchor comprising:
a connector; and
a three-dimensional projection extending from the connector,
wherein the three-dimensional projection comprises a plurality of auxetic building blocks connected together.

23. (canceled)

24. (canceled)

25. (canceled)

26. (canceled)

27. The anchor of claim 22, wherein the anchor is formed of a conductive material.

28. A method for growing tissue in a chamber, the method comprising:
filling a chamber with a scaffolding material comprising cells, the chamber with a first three-dimensional anchor located at a first end and a second three-dimensional anchor located at a second end, wherein each of the first and second three-dimensional anchors is an auxetic design; and
incubating the scaffolding material comprising the cells under conditions suitable for maturation of the cells, whereby the mature cells form tissue connecting to and between the first and second anchors.

29. The method of claim 28, further comprising providing mechanical stimulation through rotational and/or vertical movement of at least one of the first anchor and the second anchor and/or providing electrical stimulation.

30. (canceled)

31. (canceled)

32. The method of claim 28, wherein the step of incubating the scaffolding material comprising the cells under conditions suitable for maturation of the cells, whereby the mature cells form tissue connecting to and between the first and second anchors comprises:
controlling at least one of the following in the chamber: temperature, pH, dissolved oxygen and glucose; and/or
flowing liquid through the chamber.

33. The method of claim 32, and further comprising flowing liquid through the tissue.

34. The method of claim 28, wherein the cells are pluripotent stem cells such as embryonic stem cells or induced pluripotent stem cells.

35. (canceled)

36. The method of claim 28, wherein the tissue is muscle tissue and/or comprises fat tissue.

37. (canceled)

38. (canceled)

39. (canceled)

40. (canceled)

41. (canceled)

42. (canceled)
